(19) United States
(12) Patent Application Publication
(10) Pub. No.: US 2022/0240942 A1
(43) Pub. Date: Aug. 4, 2022

(54) Intra-Aneurysm Device

(71) Applicants: Universite de Montpellier, Montpellier (FR); Centre Hospitalier Universitaire de Montpellier, Montpellier (FR); Centre National de la Recherche Scientifique, Paris (FR)

(72) Inventors: Lionel Bichet, Montpellier (FR); Hervé Louche, Saint-Gély-du-Fesc (FR); Franck Jourdan, Montpellier (FR); Vincent Costalat, Saint-Gély-du-Fesc (FR)

(73) Assignees: Universite de Montpellier, Montpellier (FR); Centre Hospitalier Universitaire de Montpellier, Montpellier (FR); Centre National de la Recherche Scientifique, Paris (FR)

(21) Appl. No.: 17/629,436

(22) PCT Filed: Jul. 29, 2020
(86) PCT No.: PCT/EP2020/071434

§ 371 Date: Jan. 24, 2022

(30) Foreign Application Priority Data
Jul. 29, 2019 (EP) 19305986.2

(51) Int. Cl. A61B 17/12

(52) U.S. Cl. CPC A61B 17/12172; A61B 2017/00867; A61B 17/12118

(57) ABSTRACT

The invention relates to an intra-aneurysm device (1) for treating an aneurysm (2), comprising a woven structure (S), with meshes made of a plurality of yarns, the woven structure (S) comprising a head (5) intended to be inserted into the aneurysm (2), the head (5) being capable of significantly reducing a blood flow in the aneurysm (2), said head (5) being dome-shaped, the device (1) being characterised in that the woven structure (S) comprises, at a proximal end of said woven structure (S) a knot (4a) connected to the head (5) and in that, at its distal end opposite the proximal end, the head (5) is formed from yarn ends (6a), at least some of which are mechanically connected one another.

### TECHNICAL SCOPE OF THE INVENTION

The invention relates to the field of intra-aneurysm medical devices. More specifically, the invention relates to the flow diversion intra-aneurysm devices.

### TECHNICAL BACKGROUND

A dilation of the wall of an artery is called an “aneurysm”. The dilation causes an aneurysm sac which communicates with the artery through a narrowed zone known to practitioners as the “collar”.

The rupture of an aneurysm, or more precisely of the aneurysm sac, can have serious consequences, including death. Aneurysms are caused by several factors. It should be noted that older people have a higher prevalence. In these people, the risk of rupture is also greater, in particular, because of related pathologies such as arterial hypertension, which manifests itself in the form of increased blood pressure.

It is therefore a public health problem that needs to be treated. There are different techniques for treating an aneurysm.

Among the techniques for treating an aneurysm, the so-called “endovascular” technique differs from other techniques in that it comprises a step of treating the aneurysm with an endovascular device. In this case, the aneurysm is approached by following the path of the arteries. A carrier catheter (of an intra-aneurysm device) is introduced through the arteries and directed under fluoroscopic imaging. This can also be an extra-aneurysm device, an intra-aneurysm device or both. The device is then deposited in the aneurysm. In particular, the device is deposited in the artery carrying the aneurysm in the case of an extra-aneurysmal device or in the aneurysm in the case of an intra-aneurysm device. This isolates the aneurysm from the blood flow of the artery to prevent the aneurysm sac from rupturing due to blood pressure.

The implementation of this technique can be particularly complex depending on the technique used for the design of the device but also the structure and shape of the device, especially when the device is of the intra-aneurysm type.

Two techniques for designing cerebral endovascular devices exist.

The first technique is called “laser cut”. The expression “laser cut” according to the Anglo-Saxon terminology refers to a laser cutting method. It consists of laser cutting a tube of a shape memory alloy to create a network of open or closed cells of generally parallelepiped shape. However, the devices manufactured using this technique suffer from collapse due to the curvature of the artery, which, in addition to representing a danger to the patient, eventually requires a new intervention on the patient in order to replace them. As these interventions are very burdensome for the patient, it is advisable to limit the number.

A second technique, called “woven”, consists of creating a woven structure from one or more yarns of a shape memory alloy so as to form a network of closed cells. This technique, in contrast to the first technique described above, allows the design of devices that are repositionable, flexible and more structurally stable, allowing them to be installed permanently in the artery to be treated. The devices manufactured using the woven technique are typically tubular in shape. They can be used to treat many other pathologies.

However, the use of the woven technique for the design of intra-aneurysm devices remains very limited, and for good reason; although the use of the woven technique is well suited to the design of devices with simple shapes, for example tubular, it is much less suited to the design of devices with more complex shapes which require specific manufacturing tools and whose manufacturing time can be particularly long. The loop-like structure resulting from the configuration of the aneurysm in relation to the artery, as well as the presence of bifurcations in the immediate vicinity of the aneurysm, make the use of the woven technique for the design of intra-aneurysm devices less common, without excluding its use.

Amongst the woven techniques are the contour type devices. These devices comprise a head intended to be inserted into the aneurysm and form a woven structure made of a plurality of yarns. The head comprises a nodal end at which the yarns are crimped together and a distal end at which the yarns are “looped back and forth”. The yarns run uninterrupted at the loops and are only connected to each other at the nodal end. In addition to the ring required to crimp the yarns at this nodal end, a very large number of yarns are required to ensure the structural stability of the contour devices. These devices are therefore very cumbersome and access to some aneurysms deep in the arteries is complicated or impossible.

Among the woven techniques are “coils”. The term “coils” refers to a small metal coil used to close off a vessel. Indeed, many intra-aneurysm devices use coils to fill the aneurysm sac of the aneurysm to be treated in order to limit the flow of blood to the dome and thus the aneurysm. The operation to fill the aneurysm sac with coils is called “coiling”. This can be a relatively lengthy process as the entire aneurysm sac must be filled for the device to be fully functional.

For example, the document WO 2006/052322 describes another example of an intra-aneurysm device with a coils-type woven structure. The disclosed intra-aneurysm device comprises a stent allowing anchoring of the device in a source artery carrying the aneurysm. The stent is a term commonly used in the medical field to designate a tube of substantially circular cross-section. The anchoring is achieved by endothelialisation of the stent on the inner wall of the artery. The device comprises a head, generally in the shape of a dome, intended to fit inside the aneurysm sac. Between the dome and the stent, the device further comprises a thinned intermediate portion that delimits the dome and the stent.

However, the deployment time of this device is very long and the structure is not stable enough to allow its deployment in a shorter time.

### SUMMARY OF THE INVENTION

An objective of the invention is to solve at least one of the above problems.

To this end, an intra-aneurysm device is proposed for treating an aneurysm, comprising a woven structure, with meshes made of a plurality of yarns, the woven structure comprising a head intended to be inserted into the aneurysm, the head being capable significantly reducing a blood flow in the aneurysm, said head being dome-shaped.

The device is characterised in that the woven structure comprises at a proximal end of said woven structure a knot connected to the head and in that, at its distal end opposite said proximal end, the head is formed of yarn ends, at least some of which are mechanically connected one another.

The device according to the invention is thus configured in such a way that it can be transported to the affected zone by means of a microcatheter, the dimensions of which are much smaller than those of the device without this proving detrimental to the structural strength of the head, whose dimensions are greater than those of the microcatheter since it is intended to be inserted into the aneurysm.

Indeed, the fact that the woven structure comprises, at a proximal end of said woven structure, a knot connected to the head and that, at the distal end of said woven structure, the head is formed of yarn ends, at least some of which are mechanically connected one another, allows to preserve the structural strength of the head and to improve the structural stability of the device during the deployment phase. This avoids structural deformation of the device and the separation of the yarns from each other that would occur during passage through the microcatheter. Once the device is correctly positioned within the aneurysm, the deployment phase is completed and the device is immediately functional. No additional time is required for any filling of the aneurysm sac as is the case with coils type devices.

In addition, the device according to the invention has a very small footprint which allows it to be implanted further into the affected arteries and enables it to treat a larger number of aneurysms. This is due, on the one hand, to the fact that the knot connected to the head is formed by the woven structure itself, which avoids the need for a ring, and, on the other hand, to the fact that some of the ends of the yarns are mechanically connected one another at the end of the head opposite the knot, so that the structural stability of the device is due to these connections and not to the number of yarns, which can then be very small.

According to various features of the invention, which may be taken together or separately:
the yarn ends are mechanically connected at least two by two;
the meshes of the woven structure are denser at the proximal end of said head than at the distal end of said head;
the head has a substantially semi-spherical geometry facing outwards, the distal end of said head having a substantially circular cross-section of diameter D2;
the knot has a substantially circular cross-section with a diameter D3 smaller than the diameter of the head D2;
the number of yarns is between 4 and 250, preferably between 16 and 32;
the yarns have a diameter of between 10 μm and 500 μm;
the yarns overlap at intersection zones;
said yarns are mechanically connected at said intersection zones;
at least some of the yarn ends are mechanically connected one another through a connecting portion made of a part of a first yarn and a part of a second yarn;
the yarns are made of biocompatible material, preferably nitinol, platinum or titanium;
at least one yarn is radiopaque;
the device comprises a proximal portion for positioning the device;
said knot connects the proximal portion to the head;
the meshes of the woven structure at the knot are denser than the meshes of the woven structure at the proximal end of the head;
said knot is formed by a crimping ring connecting yarn ends to each other;
said knot is formed by yarn ends 6c welded to one another.

### BRIEF DESCRIPTION OF THE FIGURES

Further objects and features of the invention will become clearer in the following description, made with reference to the attached figures, in which:

FIG. 1 illustrates a perspective view of a device according to a first embodiment of the invention;

FIG. 2 illustrates a perspective view of a device according to a second embodiment of the invention;

FIG. 3a illustrates a side view of the device during the initiation phase of its deployment within the aneurysm;

FIG. 3b illustrates a perspective view of the device during the phase of apposition on the walls of the aneurysm;

FIG. 3c illustrates a perspective view of the device during the final phase of its deployment within the artery;

FIG. 4 illustrates a front view of the device according to the invention after deployment within a reconstructed artery;

FIG. 5 illustrates the result of a numerical simulation of deployment of the device according to the invention in front view;

FIG. 6a illustrates a close-up view of the device according to the invention during the method of connecting the yarn ends according to a first embodiment;

FIG. 6b illustrates a close-up view of the device according to the invention during the methods of connecting the yarn ends according to a second embodiment;

FIG. 6c illustrates a connecting portion of the device of FIG. 6a;

FIG. 6d illustrates a connecting portion of the device of FIG. 6b;

FIG. 7 illustrates a method of connecting yard ends;

### DETAILED DESCRIPTION OF THE INVENTION

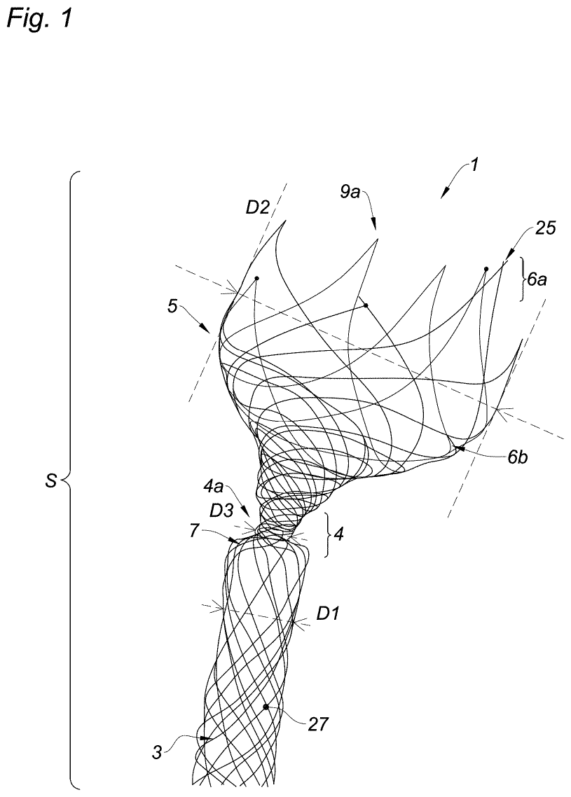

With reference to FIG. 1, the invention relates to an intra-aneurysm device 1 for the endovascular treatment of an aneurysm 2. In other words, the device 1 according to the invention is suitable and intended to be implanted within said aneurysm 2. The device 1 as illustrated in FIG. 1 is at rest.

It is recalled that an aneurysm results from the dilation of the wall of a vessel, blood, carrier. This vessel is typically an artery. The dilation causes an aneurysm sac 8 which communicates with the artery through a narrowed zone known to practitioners as the “collar”.

The aneurysm 2 in question may have any morphology. It can be a wide-necked aneurysm or not. The same applies to the carrier vessel, which can vary greatly in size depending on the part of the body involved, as other factors must be taken into account.

Furthermore, the aneurysm 2 in question may be located in any part of the body comprising an affected vessel. For example, it may be located in the aorta, making it an aortic aneurysm. It may also be located in an artery in the brain, making it a cerebral or intracranial aneurysm. Cerebral aneurysms are often difficult to access, which makes the deployment of an endovascular device and thus their endovascular treatment particularly complex and in some cases even impossible to achieve. As will be seen in the following sections, the intra-aneurysm device 1 according to the invention is particularly well suited to the treatment of cerebral aneurysms due to its easy deployment and small size.

The intra-aneurysm device 1 is more particularly a flow diversion device. The devices of this type differ from coils and intra-aneurysm cage devices essentially in that the aneurysm is treated not by filling the aneurysm sac with coils or with embolization material, as the case may be, but by treating the artery carrying the aneurysm.

The device 1 according to the invention comprises a head 5 forming a woven structure S, with meshes, made with a plurality of yarns 6.

The head 5 is intended to be inserted into the aneurysm 2. Its function is to significantly reduce the blood flow in the aneurysm 2. The arrangement of the yarns 6 at the end will be discussed in more detail below. However, it can probably be stated at this stage that the ends 6a of the yarns form a circumference of the head 5.

According to one aspect of the invention, the woven structure S comprises, at a proximal end of said woven structure S, a knot 4a connected to the head 5. This increases the structural strength of the head 5 during the deployment phase of the intra-aneurysm device 1 in the portal artery. The knot 4a allows the woven structure S to be stabilized at the head 5, i.e. between the proximal end of the woven structure S and the distal end of said woven structure S, without preventing said head 5 from being movable relative to the knot 4a.

According to an alternative embodiment of the invention, the meshes of the woven structure S at the knot 4a are denser than the meshes of the woven structure at the proximal end of the head. The density is defined here as the number of mesh(es) per unit area in arbitrary units. The meshes of the woven structure S are said to be denser because, at said knot, they are more numerous per unit area. In other words, the mesh of the woven structure S, at said knot 4a, is “fine and tight” as opposed to a loose “wider and slacker” mesh more likely to deform or lose its structure if no means are provided to preserve it. This configuration is particularly well suited to a device 1 which is provided, in addition to the head 5 and the knot 4a, with a proximal portion 3, for example a stent, as will be seen below (FIG. 1).

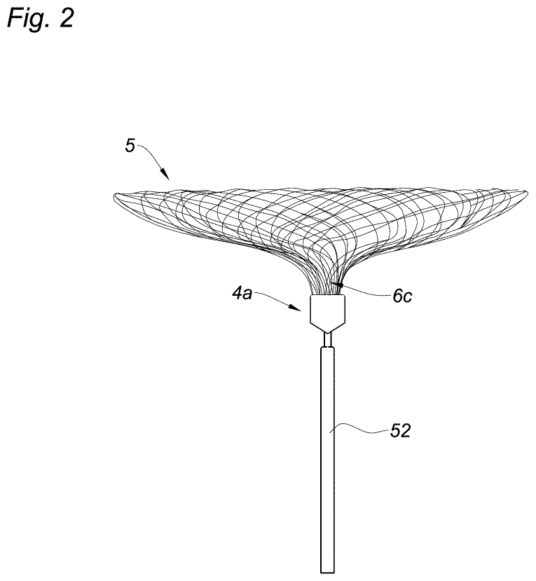

According to another embodiment of the invention, the woven structure S is formed, at said knot 4a, of connected, i.e. joined, yarn ends 6c. This configuration is particularly well suited to an intra-aneurysm device 1 comprising only a head 5 and a knot 4a (FIG. 2). Indeed, in this case, the knot 4a then connects the plurality of yarns 6. In a first configuration, said knot 4a may be formed by a crimping ring connecting yarn ends 6c to each other. Alternatively, in a second configuration, said knot 4a may be formed by yarn ends 6c welded to one another.

In either embodiment, the knot 4a has a cross-sectional diameter D3 much smaller than that of the head 5.

Furthermore, the fact that the knot 4a is connected to the head 5, at the proximal end of said woven structure S, allows to increase the manoeuvrability of the head 5 and thus reduces the intervention time. In fact, during the deployment phase, the device 1, which is initially completely inserted in the microcatheter 50, is pushed by a pusher 52 of the microcatheter 50 in the direction of the zone to be treated. The presence of the knot 4a thus prevents the device 1 from losing its structural strength under the effect of the stress exerted by the pusher 52 of the microcatheter 50 on said knot 4a and thus on the head 5 which is in its extension.

According to another aspect of the invention, at the distal end opposite said proximal end of said woven structure S, the head 5 is configured such that at least some of the yarn ends 6a are mechanically connected one another. This enables to preserve the structural strength of the device 1 during the deployment phase of the intra-aneurysm device 1 in the carrier artery. Indeed, when the device 1 is inserted into the microcatheter 50 for deployment and placement in the zone to be treated, it undergoes significant stresses due to the small dimensions of the microcatheter 50 compared to those of the intra-aneurysm device 1. Indeed, the internal diameter of the microcatheter 50 is typically between 0.4 mm and 2 mm. In such a situation, the head 5 undergoes very important constraints since it is intended to be inserted into the aneurysm 2 and has large dimensions. The connection of certain ends 6a of the yarns located at the distal end of the woven structure S allows to improve the structural stability of the head 5 and to preserve the structural strength of the device 1 by avoiding the deformation of the head 5 and the separation of the yarns 6 from each other.

Furthermore, in this configuration, there is no need for additional time for filling the aneurysm sac with coils or embolization material as is typically the case in coiled and intra-aneurysm cage devices respectively since the initial structure of the device is preserved after the deployment phase. This reduces the duration of the intervention on the patient.

Preferably, each of said ends 6a of the yarns is connected to at least one other of said ends 6a of the yarns. In other words, the ends 6a of the yarns are mechanically connected at least in pairs. The head 5 has the appearance of a water lily with conically shaped petals. In this configuration, as the ends are mechanically bonded at least in pairs continuously around the entire circumference of the head end, the structure of the meshes is preserved. Incidentally, this gives more structural strength to the head 5 and structural stability to the intra-aneurysm device 1 during the deployment phase.

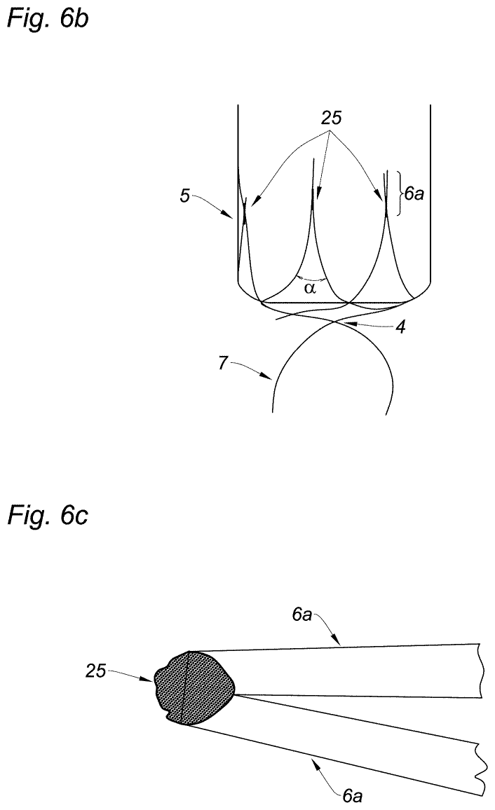

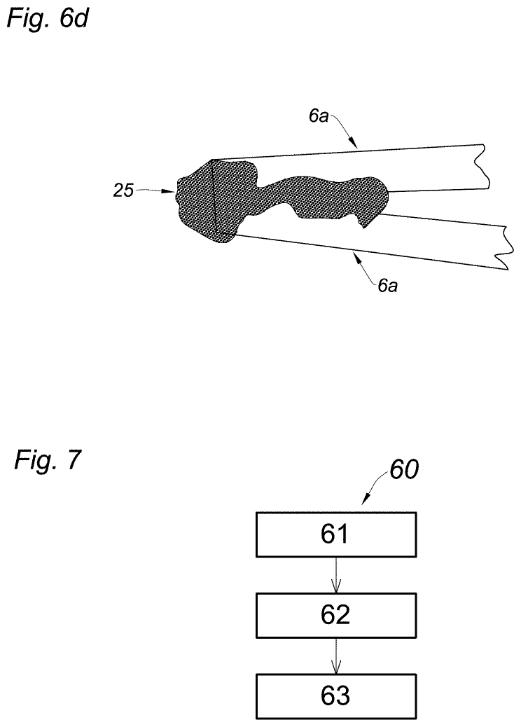

Preferably, the ends 6a of the yarns are mechanically connected to each other by means of a connecting portion 25. The portion 25 extends substantially at the peripheral portion of the yarns 6. It is not necessarily exactly the most extreme point of said yarns 6 but a portion extending from this point to a peripheral zone of said yarns 6. FIGS. 6a and 6c illustrate such a configuration.

That said, the connecting portion 25 is not necessarily a point. The connecting portion 25 may also be linear and non-point. In other words, in such a case the ends 6a of the yarns are not mechanically connected one another at a single point but in a line, or even a cord. Preferably, the length of said connecting portion 25 is between 0.05 mm and 1 mm. The mechanical connection between the ends 6a of the yarns is then stronger, and as will be seen later even without adding material. FIGS. 6b and 6d illustrate such a configuration.

Thus, with the knot 4a connected to the head 5 and the head 5 configured such that at least some of the yarn ends 6a located at the distal end of the woven structure S are mechanically connected to each other on the other hand, the structural stability of the device 1 is preserved during the phase of deployment of the device 1 in the aneurysm 2 and the intervention time on the patient is reduced. This advantageously confers on the device 1 according to the invention a reduced size in comparison with the contour type devices known in the prior art. Indeed, the structural stability of the device is due to the connections existing at the knot 4a and at least some of the ends 6a of the yarns 6 and not to the number of yarns. The latter can then be very small.

The intra-aneurysm device 1 according to the invention may have several configurations depending on whether it is at rest, in the deployment phase or in use. The ability of the device 1 to change from one configuration to another is due, on the one hand, to the properties of the material used for its design and, on the other hand, to its structure based on woven micro-yarns. However, as we shall see in detail in the following sections, this ability of the device 1 according to the invention to change from one configuration to another does not affect its good structural performance, in particular during the deployment phase.

With reference to FIG. 1, the following describes the configuration of the intra-aneurysm device 1 according to a first embodiment of the invention at rest, i.e. the configuration of the device 1 in the absence of any pressure on said device 1.

The device 1 according to this first embodiment of the invention may comprise in addition to the head 5, a proximal portion 3, which is for example a stent. The proximal portion 3, the knot 4a and the head 5 then form the woven structure S.

In this particular embodiment, the advantages of the intra-aneurysmal technique are combined with those of conventional stents.

As mentioned above, the woven structure S is made of a plurality of yarns 6. The yarns 6 are arranged so as to form a plurality of cells of substantially parallelepiped shape. Each cell or mesh comprises a central zone devoid of material, i.e. not covered by the yarns 6, and a periphery delimited by the yarns 6. The cells are thus said to be “closed”. Depending on the surface area of the cells, the woven structure S is associated with a fine mesh or a wide mesh. In this case, a fine mesh comprises cells occupying a smaller surface area compared to a wide mesh. It should be noted that, in this particular embodiment, the proximal portion 3, the head 5 and the knot 4a may be formed with the same yarns 6, which contributes to improving the structural strength of the intra-aneurysm device 1, but they do not necessarily have the same type of mesh.

The yarns 6 have millimetre longitudinal dimensions and micrometre diametrical dimensions. More precisely, the yarns 6 have a diameter of between 10 and 500 μm. This diameter is advantageously less than or equal to 50 μm for cerebral aneurysms. However, for thoracic aneurysms it is approximately 500 μm. Preferably, the number of yarns 6 is between 4 and 250 depending on the dimensions of the device, and indeed the dimensions of the aneurysm and the carrier artery. Even more preferably, the number of yarns varies between 16 and 32. Such a number of yarns 6 advantageously facilitates the insertion of the intra-aneurysm device 1 into a microcatheter 50 used to deploy the device 1 in the arterial zone affected by the aneurysm. This also allows for a woven structure S of sufficient flexibility and manoeuvrability for insertion into the most difficult accessible parts of the body. However, it should be noted that the number of yarns 6 depends substantially on the diameter of the yarns. Thus, if the diameter of the yarns 6 is 100 μm, the number of yarns cannot exceed 4. On the other hand, if the diameter of the yarns 6 is 25 μm, the number of yarns can be much higher, for example 16.

In this respect, the intra-aneurysm device 1 is advantageously made of a nickel and titanium alloy commonly known as nitinol. In addition to the workability that this material offers, it also has excellent shape memory and very good elasticity, which allows it to conform to the morphology of the zone in which it is located.

Advantageously, at least one yarn is radiopaque. This allows the yarn to be viewed on a screen during the procedure, which also allows the opening status of the device 1 to be observed.

The proximal portion 3 is suitable and intended for positioning the intra-aneurysm device 1 in the portal artery. In this particular embodiment, the proximal portion 3 has at rest a substantially circular cross-section of diameter D1 suitable for anchoring/positioning the intra-aneurysm device 1 in the artery affected by the aneurysm 2. The proximal portion 3 thus has a substantially tubular shape.

The knot 4a is, in turn, advantageously positioned at an intermediate portion 4 of the device 1. The intermediate portion 4 extends from an upper end 7 of the proximal portion 3 to a base of the head 5. The knot 4a has a circular cross-section with a diameter, D3, which is smaller than the diameter D1 of the proximal portion 3, without preventing the flow of blood through said knot 4a. For example, the diameter D3 of knot 4a may be twice the diameter D1 of the proximal portion 3. This allows for good blood flow through the arteries underlying the aneurysm 2.

As mentioned above, the proximal portion 3, the knot 4a and the head 5 are advantageously formed with the same yarns 6. However, the mesh size of the woven structure S at knot 4a is narrower. This allows, in addition to the advantages seen above, to dispense with the use of an additional clamping means such as a ring.

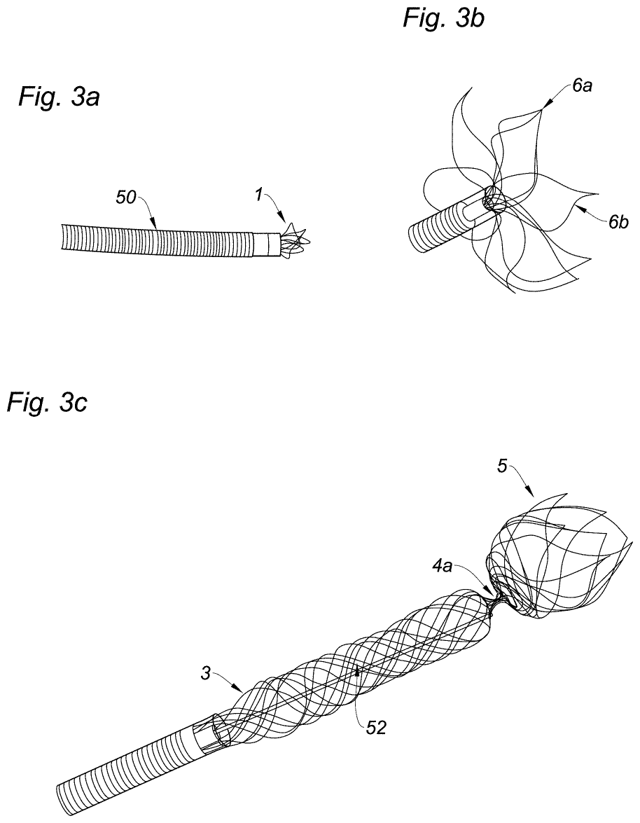

With reference to the first (FIG. 1) and second (FIG. 2) embodiments of the invention, the head 5 has a concave dome shape with the concavity 9a facing outwards. In other words, the head 5 is in the form of a truncated half-sphere or sphere or a truncated flare or ovoid facing outwards. The distal end of the head 5 has a substantially circular cross-section with a diameter D2 generally increasing towards its end. Indeed, at its distal end, the diameter of the head 5 decreases slightly due to the slight inward curvature of the petals. It is at a section between its distal end and a middle part that the diameter D2 of the head 5 is maximum. However, overall it is much larger than the diameter at the knot, and even larger than the diameter D1 of the proximal portion 3 in the embodiment shown in FIG. 1. Once the device 1 is installed on a patient, the concavity 9a faces the aneurysmal pocket 8 as shown in FIG. 3. However, this does not prevent the circumference of the free end of the head 5 from being coarse and sinuous, i.e. it is not regular or straight.

At the proximal end of said head, the meshes of the woven structure S are denser than at the distal end of said head, i.e. the meshes are looser. Thus, the meshes of the head 5 have increasing dimensions the further they are from the intermediate portion 4, in particular from the knot 4a. In other words, at its end, the mesh of the head 5 is therefore much wider than the mesh of the head 5 in the immediate vicinity of the intermediate portion 4. It is also wider than that of the proximal portion 3, of the intermediate portion 4 and thus of the knot 4a. In a particularly advantageous manner, such a mesh also allows to promote the pressing of the head 5 against the walls of the aneurysm 2 when the intra-aneurysm device 1 is in use, i.e. after deployment. The use of nitinol yarns 6 certainly contributes to such plating, but to a much lesser extent, nitinol having a relatively low radial force.

It may also be pointed out that, as the mesh of the woven structure S at the head 5 is relaxed, the risk of the mesh shrinking is considerably reduced. Incidentally, this reduces the risk of delayed rupture of the aneurysm 2. Indeed, a possible retraction of the mesh of the head 5 would have as a consequence to leave a free space between the head 5 and the aneurysm 2 in which the blood could circulate by exerting without any constraint a pressure on the walls of the aneurysm 2, which in the long term would cancel the effects. In addition, the loose mesh at the head 5 allows for a substantial increase in the flexibility of the head 5, which advantageously allows for the treatment of aneurysms of more varied geometries and dimensions and thus for the treatment of a greater number of aneurysms.

This being said, the interest of the solution of the invention appears even better considering the less dense mesh of the woven structure S at the level of the head 5. Indeed, the fact that knot 4a is connected to the head 5 stabilises the mesh at the proximal end of the woven structure, i.e. on the knot 4a side of the head 5. At the same time, at the distal end of the woven structure, i.e. on the side of the head 5 opposite the knot 4a, the presence of connections between some of the ends 6a of the yarns prevents the initial mesh from being completely altered by the disassembly of the meshes of which it is composed.

With reference to FIGS. 2a to 2c, the main deployment phases of the intra-aneurysm device 1 are illustrated.

FIG. 3a shows the intra-aneurysm device 1 in the initial phase of deployment. In this configuration, the head 5 is barely visible, as the device 1 is almost completely inserted into the microcatheter 50. The ends 6a of the yarns are visible.

FIG. 3b illustrates the intra-aneurysm device 1 in a more advanced phase of deployment. In this configuration, the intermediate portion 4, the knot 4a and the proximal portion are still not visible. The microcatheter 50 continues to exert pressure on the portion of the head 5 located near the intermediate portion. Nevertheless, the petal-like structure resulting from the bonding of at least some of the ends 6a of the yarns to each other is more clearly visible.

Disjointed portions 6b of the yarns 6 appear less close to each other compared to the configuration they adopt when the device 1 is at rest (FIG. 1). Even when subjected to the stress generated by the separation of the disjointed portions 6b, the connections 25 are maintained. This is due to the process of connecting the ends 6a of the yarns, which will be described in more detail later.

In this respect, if some of the ends 6a of the yarns are mechanically connected, they should only be connected during the deployment phase. Indeed, the loss of structural strength is only harmful if it occurs before the head 5 has been correctly positioned within the aneurysm 2. However, it is precisely during the deployment phase that the risk of loss of structural strength is highest due to the difference in size between the intra-aneurysm device 1 and the microcatheter 50, which, as a reminder, has dimensions of between 0.4 and 2 mm. This being recalled, if the ends 6a of the yarns become disconnected from each other after deployment of the device 1, this does not affect the operation of the device 1.

FIG. 3c shows the intra-aneurysm device 1 in the final phase of deployment. In this configuration, almost the entire device 1 is deployed. The proximal portion 3, the knot 4a and the head 5 are clearly visible. No more pressure is exerted on the head 5 so that it returns to its resting configuration. The structural strength of the head 5 has been preserved since the mesh of the woven structure S is intact at the head 5. This would not have been the case if the knot 4a was not connected to the head 5. Indeed, in this case, under the effect of the thrust of the pusher 52 of the microcatheter, the knot 4a and then the head 5 would have been deformed. This would also not have been the case if some of the ends 6a of the yarns had not been mechanically connected one another. Indeed, in this case, under the effect of the push of the pusher 52 of the microcatheter and the pressure exerted by the internal walls of the microcatheter 50 (as illustrated in FIG. 2b), the mesh of the woven structure S would have disintegrated.

In addition, the proximal portion 3, whose diameter D1 is greater than the diameter of the microcatheter 50 and around which pressure was exerted, also returned to its resting configuration.

It should be noted that other connections may be provided to further improve the structural stability of the device 1. The woven structure S may comprise, at certain intersections between the yarns 6, connecting points 27 at which the yarns 6 are mechanically connected. Said connection points 27 are located at one or more suitable positions in the woven structure S, i.e. optionally in the mesh of the proximal portion 3, the mesh of the intermediate portion 4 or in the mesh of the head 5. There is less interest in the mesh of the intermediate portion 4, which is advantageously relatively fine. That said, at the proximal portion 3 and the head 5, this can be very advantageous in order to further stabilise the woven structure S when the device passes through the microcatheter 50.

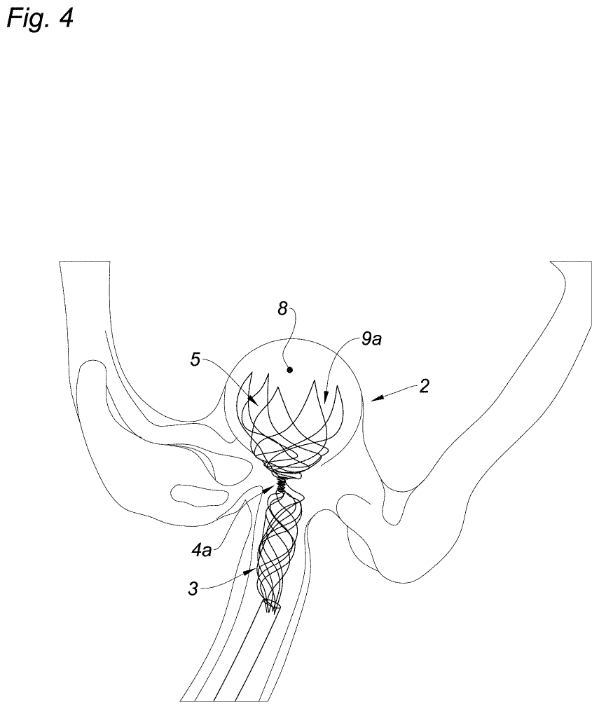

With reference to FIG. 4, the intra-aneurysm device 1 is illustrated during the final phase of its deployment in a reconstructed aneurysm 2 for the purposes of the operated tests. The reconstructed aneurysm 2 is here made of silicone. The device 1 has retained its original structure. The head 5 is correctly positioned within the aneurysm 2. It rests on the collar of the aneurysm 2 and its concavity 9a faces the aneurysm sac 8. The intermediate portion 4 is located at the level of the aneurysm collar.

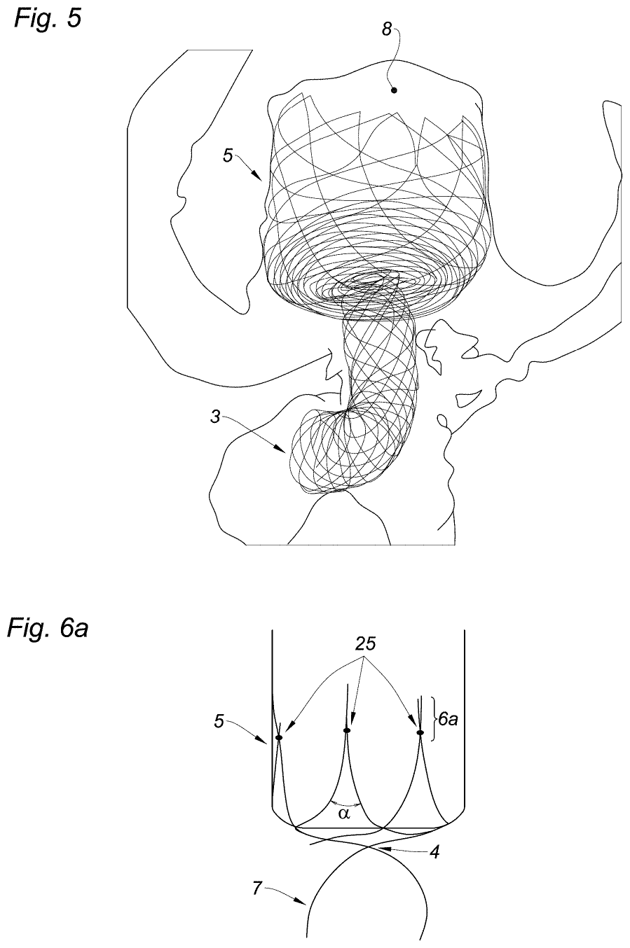

With reference to FIG. 5, the result of a numerical simulation of the deployment of the intra-aneurysm device 1 according to the invention is illustrated. These simulations, in particular the simulation illustrated in FIG. 4b, allow to better distinguish one end of the proximal portion 3.

In the following, methods 60 for joining the ends 6a of the yarns one another are described.

It should be noted that before implementing this method, the intra-aneurysm device 1 comprising the woven structure S as described above is provided with the only particularity that the ends 6a of the yarns are not joined.

With reference to FIG. 6, a close-up view of the intra-aneurysm device 1 according to the invention is illustrated for the purpose of understanding the steps of the process of joining said ends 6a of said yarns one another.

In a first step 61, two yarns 6 whose ends 6a are to be joined are brought together so that their ends 6a are substantially parallel.

By bringing the two yarns 6 together, a portion of substantially triangular shape is formed, an apex of which corresponding to a point of bringing together (which, once finally welded, is none other than the previously described connecting portion 25) of said ends 6a has an angle α (visible in FIGS. 6a and 6b).

Preferably, within the device at rest, the angle α is as closed as possible, i.e. the angle α is not open. The head 5 can thus be retracted as little as possible when inserted into the microcatheter 50 without concomitantly losing the connections between the ends 6a of said yarns. Indeed, by securing the ends 6a with an open angle, the radial stresses exerted by the walls of the microcatheter 50 on the head 5 would cause the ends 6a to become unsecured before the end of the deployment phase.

During a second step 62, some of the ends 6a are advantageously connected one another by means of a fixing method.

Advantageously, this connection can be carried out by welding the ends 6a without adding material. In this case, the two ends 6a are fused to one another by welding, using the material at the ends 6a as the material for the fusion. The welding without the addition of material reduces the size of the device 1, making it easier to insert into the zone affected by the aneurysm. The material used here is preferably a nickel-titanium or nitinol alloy. As a reminder, the woven structure S can advantageously be made of nitinol. The advantages of such a material are mentioned in the previous sections.

If the connecting portion 25 is in the form of a line or a cord, as mentioned above, two yarn ends 6a are placed in parallel and fused to one another by welding using, as in the previously described configuration, the material of the ends 6a. Preferably, this fusion is carried out over a length of between 0.05 mm and 1 mm. Here again, no additional material is required, thus improving the strength of the mechanical connection between two ends 6a without increasing the size of the device.

Such welds could for example be implemented by means of a SweetSpot® resonator. Such a device advantageously allows welds of less than 0.1 mm in size to be made.

However, the connections at the ends can also be made by welding with the addition of material. In this case, instead of using the material from which the ends 6a are made, the weld is made with a molten metal addition at the point of connection. In this respect, a blue single strand of 1 to 2 mm can be used.

The third step 63 is a cooling step. Once the welding point has cooled down completely, the two ends 6a are permanently connected. The device is then ready for use.

As an alternative to steps 61 to 63 relating to a welding method, a method of crimping the ends 6a of the yarns may also be provided.

1. An intra-aneurysm device (1) for treating an aneurysm (2), comprising a woven structure (S), with meshes made of a plurality of yarns, the woven structure (S) comprising a head (5) intended to be inserted into the aneurysm (2), the head (5) being capable of significantly reducing a blood flow in the aneurysm (2), said head (5) being dome-shaped, the device (1) being characterised in that the woven structure (S) comprises, at a proximal end of said woven structure (S) a knot (4a) connected to the head (5) and in that, at its distal end opposite the proximal end, the head (5) is formed from yarn ends (6a), at least some of which are mechanically connected one another.

2. The device (1) according to claim 1, wherein the yarn ends (6a) are mechanically connected at least two by two.

3. The device (1) according to one of claim 1, wherein the meshes of the woven structure (S) are denser at the proximal end of said head than at the distal end of said head.

4. The device (1) according to claim 1, wherein the head (5) has a substantially semi-spherical geometry facing outwards, the distal end of said head (5) having a substantially circular cross-section of diameter (D2), and the knot (4a) having a substantially circular cross-section with a diameter (D3) smaller than the diameter (D2) of the head (5).

5. The device (1) according to claim 1, wherein the number of yarns is between 4 and 250, preferably between 16 and 32.

6. The device (1) according to claim 1, wherein the yarns (6) have a diameter between 10 μm and 500 μm.

7. The device (1) according to claim 1, wherein the yarns (6) overlap at intersection zones (27), said yarns (6) being mechanically connected at said intersection zones (27).

8. The device (1) according to claim 1, wherein at least some of the yarn ends (6a) are mechanically connected one another through a connecting portion (25) made of a part of a first yarn (6) and a part of a second yarn (6).

9. The device (1) according to any one of the preceding claims, wherein the yarns (6) are made of biocompatible material preferably nitinol, platinum or titanium.

10. The device (1) according to claim 1, wherein at least one yarn (6) is radiopaque.

11. The device (1) according to claim 1 comprising a proximal portion (3) for positioning the device (1), said knot (4a) connecting the proximal portion (3) to the head (5).

12. The device (1) according to claim 1, wherein the meshes of the woven structure at the knot (4a) are denser than the meshes of the woven structure at the proximal end of the head.

13. The device (1) according to claim 1, wherein said knot (4a) is formed by a crimping ring connecting yarn ends (6c) to each other.

14. The device (1) according to claim 1, wherein said knot (4a) is formed by yarn ends (6c) welded to one another.
